(19) United States
(12) Patent Application Publication
(10) Pub. No.: US 2015/0011913 A1
(43) Pub. Date: Jan. 8, 2015

(54) DETERMINATION DEVICE, DETERMINATION METHOD, AND COMPUTER PROGRAM PRODUCT

(71) Applicant: KABUSHIKI KAISHA TOSHIBA, Tokyo (JP)

(72) Inventors: Sawa FUKE, Kawasaki-shi (JP); Takuji SUZUKI, Yokohama-shi (JP); Kanako NAKAYAMA, Tokyo (JP)

(21) Appl. No.: 14/321,044

(22) Filed: Jul. 1, 2014

(30) Foreign Application Priority Data
Jul. 2, 2013 (JP) 2013-139295

(51) Int. Cl. A61B 5/11

(52) U.S. Cl. CPC A61B 5/1121; A61B 5/1116
USPC 600/587

(57) ABSTRACT

According to an embodiment, a determination device includes a first calculator, a second calculator, a determination unit, and an output unit. The first calculator is configured to calculate inclination information that indicates inclination of an attachment surface to which a body unit is to be attached, based on acceleration of the body unit measured during a first period. The second calculator is configured to calculate difference information that indicates a difference between the inclination information stored in a storage and inclination information newly calculated by the first calculator. The determination unit is configured to determine that an attached state of the body unit with respect to the attachment surface changes when the difference information continuously indicates a difference not less than a predetermined value during a second period. The output unit is configured to output information based on a determination result obtained by the determination unit.

### CROSS-REFERENCE TO RELATED APPLICATION

This application is based upon and claims the benefit of priority from Japanese Patent Application No. 2013-139295, filed on Jul. 2, 2013; the entire contents of which are incorporated herein by reference.

### FIELD

An embodiment described herein relates generally to a determination device, a determination method, and a computer program product.

### BACKGROUND

Conventionally, it is known that there is a correlation between a pulse wave propagation velocity and time calculated from a wave form of a pulse wave or electrocardiogram measured at two points in an artery in a living body, and a blood pressure value of the living body. Accordingly, a technique of measuring the pulse wave propagation velocity with a sensor device attached to a body of a subject and of continuously estimating blood pressure has been developed for purposes of physical condition and health management, and follow-up observation of an illness.

Currently, various sensor devices for detecting signals such as, for example, a living body signal, have been downsized and equipped with a wireless function, leading to reduction in a burden when a sensor device is attached to the subject. In a future daily life, it is assumed that the subject attaches, removes, and operates a sensor device by himself or herself, and continuously measures a body surface potential difference, pulse wave signal, and living body sound of an electrocardiogram, electromyogram, etc. for a long period of time to estimate blood pressure, a posture, a heartbeat sound, a respiratory sound, etc.

Meanwhile, for measurement of the body surface potential difference and the pulse wave signal of an electrocardiogram, an electromyogram, etc., since a signal of interest differs from purpose to purpose, a preferable attachment position is determined for each purpose. That is, a measurement value may become invalid when the attachment position of a sensor device is mispositioned from a preferable attachment position. Accordingly, when the sensor device is mispositioned from a correct attachment position or when the sensor device is affixed on a position different from the last attachment position at a time of removal and attachment, the subject needs to recognize that effect and to return the attachment position of the sensor device to the correct attachment position.

However, at a time of measurement of a living body signal, for example, even when an identical signal of an identical device is used, the correct attachment position differs from purpose to purpose of determination and diagnosis. Therefore, it is difficult to predefine an only one correct attached state, and it is difficult to accurately determine whether a sensor device is mispositioned from an attachment position for each purpose such as determination and diagnosis. It is difficult to make such a determination when measurement and analysis are performed with sensor devices being continuously attached to a plurality of regions on a body surface, such as, for example, blood pressure estimation technique based on a pulse wave propagation velocity, electrocardiogram, muscle activity measurement, etc.

### BRIEF DESCRIPTION OF THE DRAWINGS

FIG. 1 is a block diagram illustrating a hardware configuration of a determination device according to an embodiment;

FIG. 2 is a functional block diagram illustrating an overview of a function of the determination device according to an embodiment;

FIG. 3 is a schematic diagram illustrating an example of a state in which the determination device is attached to a trunk of a subject;

FIG. 4 is a flow chart illustrating an example of processing performed by the determination device according to an embodiment;

FIG. 5 is a schematic diagram illustrating an attachment position of the determination device attached to a living body; and

FIG. 6 is a graph illustrating a specific example of difference information caused by misposition of the attachment position illustrated in FIG. 5.

### DETAILED DESCRIPTION

According to an embodiment, a determination device includes a first measurement unit, a first calculator, a storage, a second calculator, a determination unit, and an output unit. The first measurement unit is fixed to a body unit to be attached to an attachment surface, and is configured to measure acceleration in at least one direction. The first calculator is configured to calculate inclination information that indicates inclination of the attachment surface based on the acceleration measured by the first measurement unit during a first period. The storage is configured to store therein the inclination information calculated by the first calculator. The second calculator is configured to calculate difference information that indicates a difference between the inclination information stored in the storage and inclination information newly calculated by the first calculator. The determination unit is configured to determine that an attached state of the body unit with respect to the attachment surface changes when the difference information continuously indicates a difference equal to or greater than a predetermined value during a second period. The output unit is configured to output information based on a determination result obtained by the determination unit.

A technique of continuously estimating blood pressure has been developed for purposes of physical condition and health management, and follow-up observation of an illness. In addition, a technique of improving accuracy of blood pressure estimation has also been developed based on a characteristic of amplitude and inclination in a predetermined section of a wave form of a pulse wave, and a characteristic of an acceleration pulse wave obtained by performing second order differentiation on the pulse wave.

A relationship between a pulse wave propagation velocity and blood pressure depends on properties such as a blood vessel distance between measurement points, elastic modulus of a blood vessel wall, and a blood vessel diameter. That is, it is desirable to measure a living body signal at an identical position (identical condition) of a subject as much as possible. For example, in order to estimate a blood pressure value more accurately, when an attached state of a sensor device changes, it is necessary to detect and recognize that it is difficult to apply blood pressure value estimation means.

A wave form of a pulse wave and an electrocardiogram is used not only for estimation of blood pressure but also as an index of a cardiovascular disease. Different attachment position and device direction of a photoelectric sensor and pressure sensor for pulse wave measurement, or of an electrode for electrocardiogram measurement will lead to a different measurement result. That is, in the same manner as a case of estimation of a blood pressure value, when the attached state of the sensor device changes, it is desirable to detect and recognize the change in the attached state.

In addition, it is possible to estimate a state of the subject from a characteristic of a living body sound, for example, from a respiratory sound, a phonocardiogram, a mastication sound, by disposing a microphone on a living body surface. In a case of a phonocardiogram, for example, since an optimal attachment position of the microphone differs depending on cardiopathy to measure, it is desirable to recognize that the attached state differs.

In addition, a technique of analyzing quantity of exercise, calorie consumption, and posture of the subject from muscle activity measured with the sensor device attached to a body is widely proposed. When a characteristic of the exercise is measured and analyzed, it is desirable to correctly attach the measurement electrode at an identical position (identical condition) of the subject or at a predetermined position specified by a system as much as possible. Therefore, the technique of detecting the change in the attached state of the sensor device is important in a same manner as the case of estimation of a blood pressure value.

### Embodiment

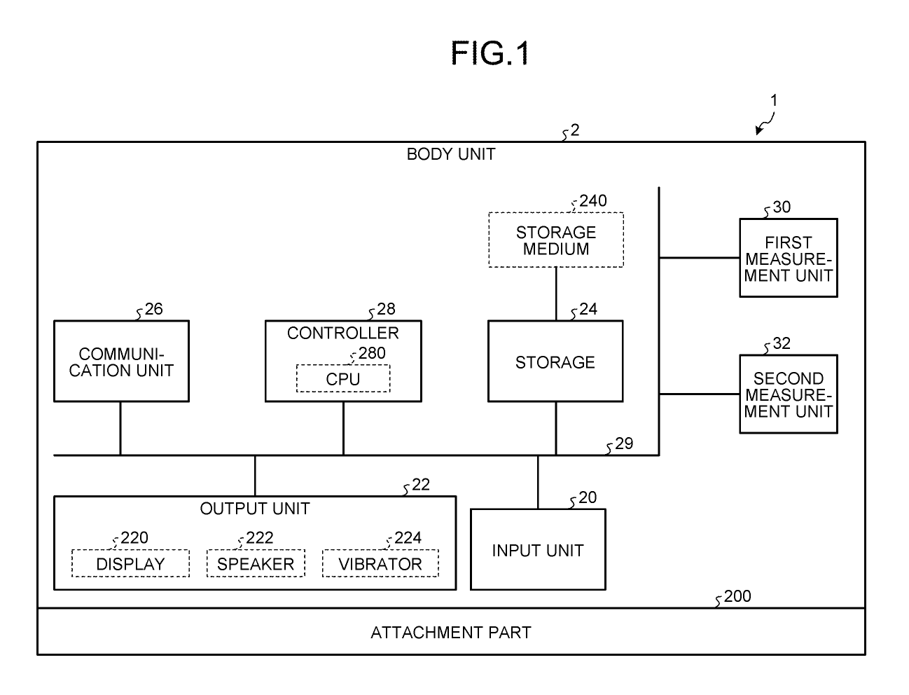

With reference to the accompanying drawings, an embodiment of a determination device will be described in detail below. FIG. 1 is a block diagram illustrating a hardware configuration of a determination device 1 according to the embodiment. The determination device 1 includes, for example, a body unit 2 that functions as a living body signal measuring device, and an attachment part 200 for attaching the body unit 2 to a subject. The attachment part 200 is made of, for example, an adhesive member, and attaches the body unit 2 to a body surface of the subject. The attachment part 200 may be a belt, etc. and may be a mounting part that mounts the body unit 2 on clothes, etc. of the subject. That is, the attachment part 200 removably attaches the body unit 2, for example, to a body surface of the subject whose living body signal and body movement are measured, or to an attachment surface such as a surface in a position substantially identical to a surface of the subject.

The body unit 2 includes an input unit 20, an output unit 22, a storage 24, a communication unit 26, a controller 28, a first measurement unit 30, and a second measurement unit 32. Each of the units constituting the body unit 2 is connected to each other via a bus 29.

The input unit 20 is, for example, an input key, a switch, or the like, and receives an input to the body unit 2 from the subject. The output unit 22 includes a display 220 such as a liquid crystal panel, a speaker 222 that outputs voice and the like, and a vibrator 224 that generates vibration. The output unit 22 outputs a result of processing operation of the body unit 2 and the like with at least one of display, sound, and vibration. The input unit 20 and the display 220 may be integrated by a touch panel or the like.

The storage 24 includes unillustrated devices such as a read only memory (ROM) and a random access memory (RAM). The storage 24 stores a program executed by the controller 28, data used when the controller 28 executes the program, and the like. In addition, a storage medium 240, such as a memory card capable of exchanging a program and data with the storage 24, may be detachably provided in the body unit 2.

The communication unit 26 is a general-purpose interface that performs communication with an external device (such as a computer). The communication unit 26 performs, for example, cable communication, long-distance wireless communication, or proximity wireless communication. The communication unit 26 may receive an input operation from the subject in place of the input unit 20 by performing wireless communication with an external device and receiving a command from the external device. Similarly, the communication unit 26 may cause an external device to output a result of processing operation by performing wireless communication with the external device and transmitting a result of processing operation to the external device. That is, the communication unit 26 also functions as an output unit that outputs information through communication.

The controller 28 includes, for example, a CPU 280, and controls each unit that constitutes the determination device 1.

The first measurement unit 30 includes an acceleration sensor that continuously measures acceleration in at least one direction. The first measurement unit 30 may also measure acceleration in a gravity direction. In the present embodiment, the first measurement unit 30 includes a 3-axis acceleration sensor with a measurement axis being fixed to the body unit 2 and a sampling frequency being 128 Hz.

The second measurement unit 32 includes, for example, an electrode that measures a body surface potential difference, a photoelectric sensor and pressure sensor that measure a pulse wave signal, a temperature sensor, an audio microphone, and a pulse oximeter. That is, the second measurement unit 32 includes a sensor device that measures a living body signal, such as an electrocardiogram, a pulse wave, a body temperature, a living body sound, and a blood oxygen level.

The configuration of the determination device 1 is not limited to the configuration illustrated in FIG. 1. For example, the determination device 1 may include the first measurement unit 30, the second measurement unit 32, the controller 28, the storage 24, and the communication unit 26. The determination device 1 may be configured to output information based on a result measured by the first measurement unit 30 and the second measurement unit 32 to an external display device or the like via the communication unit 26.

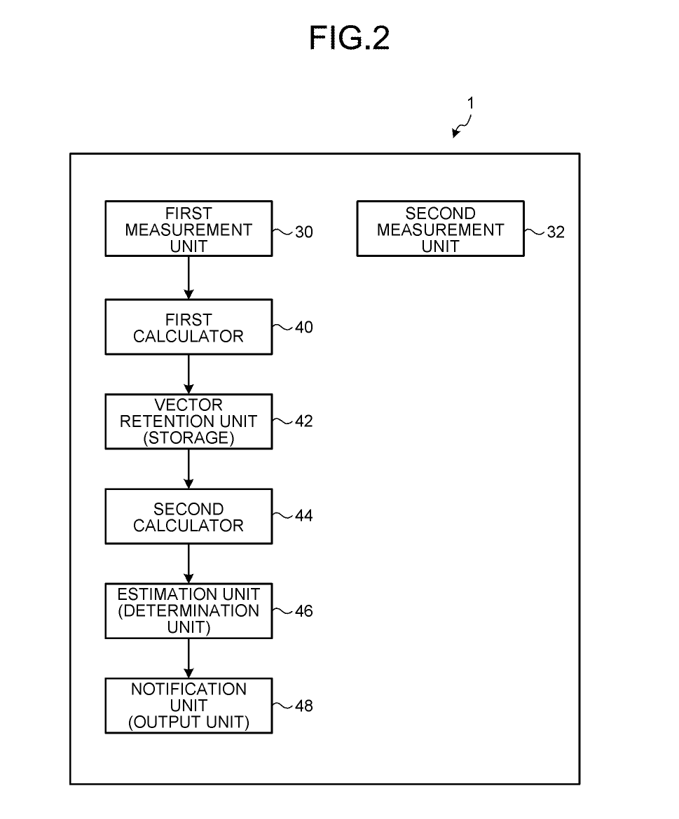

Next, a function of the determination device 1 will be described. FIG. 2 is a functional block diagram illustrating an outline of a function that the determination device 1 includes. As illustrated in FIG. 2, the determination device 1 includes the first measurement unit 30, the second measurement unit 32, a first calculator 40, a vector retention unit (storage) 42, a second calculator 44, an estimation unit (determination unit) 46, and a notification unit (output unit) 48. The first measurement unit 30 and the second measurement unit 32 illustrated in FIG. 2 correspond to the first measurement unit 30 and the second measurement unit 32 illustrated in FIG. 1, respectively. The vector retention unit (storage) 42 may be identical to the storage 24 illustrated in FIG. 1. The notification unit (output unit) 48 may be the output unit 22 illustrated in FIG. 1, and may be the communication unit 26.

The first calculator 40 calculates, for example, inclination information that indicates inclination of a body surface (attachment surface) of the subject based on acceleration measured by the first measurement unit 30 during a predetermined time (first period). For example, the first calculator 40 calculates a vector (body surface vector) that indicates inclination of the body surface to which the body unit 2 is attached as the inclination information by using the acceleration (acceleration signal) measured by the first measurement unit 30 during the first period.

The first calculator 40 calculates the inclination information that indicates the inclination of the attachment surface, after, for example, performing a filtering process on the acceleration measured by the first measurement unit 30 during the first period, the filtering process eliminating variation in the acceleration caused by a respiration or temporary movement of the subject. Examples of filters used by the first calculator 40 include, but are not limited to, FFT, IIR type LPF, or a moving-average filter.

The vector retention unit 42 stores (retains) the inclination information calculated by the first calculator 40.

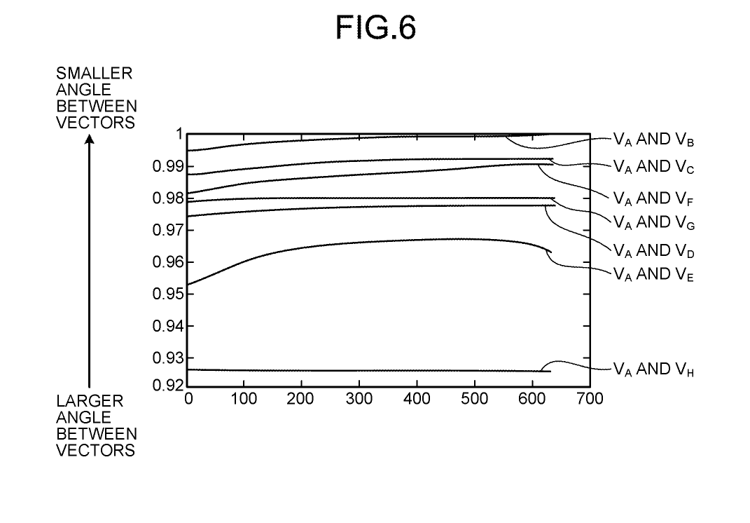

The second calculator 44 calculates difference information that indicates a difference between the inclination information stored in the vector retention unit 42 and inclination information newly calculated by the first calculator 40. For example, the second calculator 44 calculates at least one of an inner product, an angle, and a cosine value of the angle (see FIG. 6) as difference information, the inner product, the angle, and the cosine value of the angle being formed by the vector stored in the vector retention unit 42 and a vector newly calculated by the first calculator 40. In order to calculate the difference information, the second calculator 44 may use a difference of each factor of the vector, and may further use another kind of information such as a vector length.

The estimation unit 46 estimates (determines) that an attached state (installation condition) of the body unit 2 with respect to the attachment surface changes when the difference information calculated by the second calculator 44 continuously indicates a difference equal to or greater than a predetermined value for a predetermined time (second period). A change in the attached state of the body unit 2 with respect to the attachment surface refers to, for example, a case where an attachment position of the body unit 2 attached by the attachment part 200 is mispositioned with respect to the body surface of the subject.

The notification unit 48 includes, for example, the output unit 22 or the communication unit 26. The notification unit 48 outputs information based on a determination result obtained by the estimation unit 46. For example, when the attached state of the body unit 2 changes with respect to the attachment surface, the notification unit 48 outputs at least one of display, sound, and vibration that indicate the change. The notification unit 48 may notify the subject of information through communication, and may transmit information to a third party at a distant position for notification.

Functions included in the determination device 1 are not limited to those configured by a form illustrated in FIG. 2 in the same manner as the hardware configuration described above. For example, the first calculator 40, the vector retention unit 42, the second calculator 44, the estimation unit 46, and the notification unit 48 may be provided in an external device that receives a measurement result of the first measurement unit 30 and the second measurement unit 32.

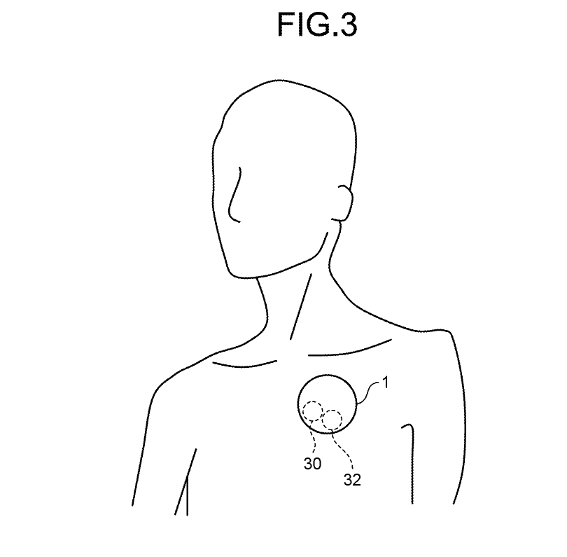

FIG. 3 is a schematic diagram illustrating an example of a state in which the determination device 1 is attached to a trunk of the subject. As illustrated in FIG. 3, the determination device 1 has at least the first measurement unit 30 (and the second measurement unit 32), is attached to the attachment surface, and outputs information based on the measurement result.

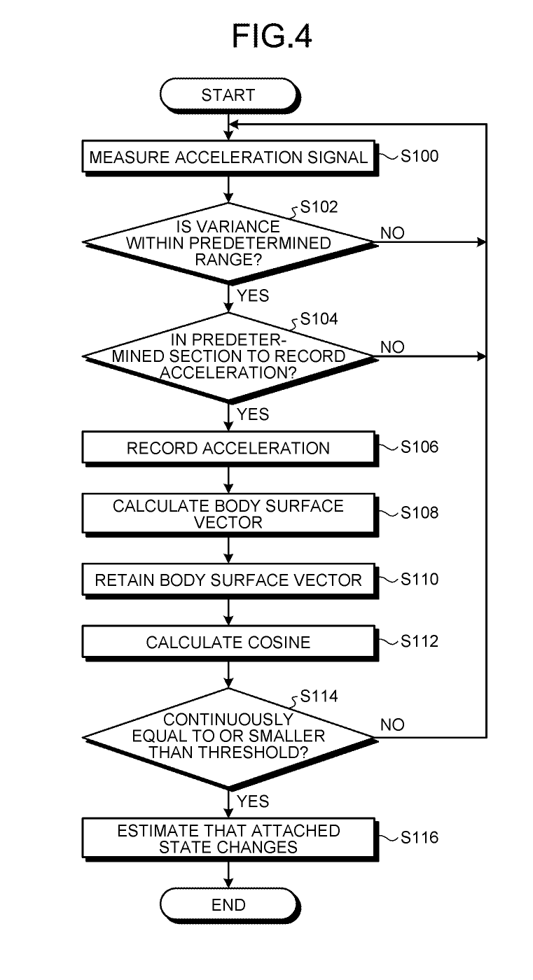

Next, processing performed by the determination device 1 will be described. FIG. 4 is a flow chart illustrating an example of processing performed by the determination device 1 according to the embodiment.

When the determination device 1 is attached to the subject, and is turned on, for example, the first measurement unit 30 starts measurement of the acceleration signal in step 100 (S100).

In step 102 (S102), the controller 28 determines whether variance of the acceleration measured by the first measurement unit 30 is within a predetermined range. The controller 28 returns to processing of S100 when the variance of the acceleration is not within the predetermined range (S102: No). The controller 28 goes to processing of S104 when the variance of the acceleration is within the predetermined range (S102: Yes).

In step 104 (S104), the controller 28 determines whether the determination device 1 is in a state where a change of the attached state thereof is to be estimated. Specifically, the controller 28 determines whether the determination device 1 is in a predetermined section (first period) in which an acceleration value needs to be recorded to calculate inclination information about the body surface to which the determination device 1 is attached using a continuous value of the acceleration measured by the first measurement unit 30. For example, when the variance of the acceleration value is equal to or smaller than a predetermined threshold, the controller 28 estimates that the subject to which the determination device 1 is attached is continuously in a stationary state. The controller 28 then determines that the determination device 1 is in the predetermined section in which the acceleration value needs to be recorded. This is because the subject is in a stationary state such as in a supine position when, for example, the second measurement unit 32 measures a living body signal such as a pulse wave and an electrocardiogram.

Hereinafter, the predetermined section (first period) refers to time in a predefined length in which the subject to which the determination device 1 is attached is estimated to be continuously in a stationary state, such as a case where variance of an acceleration value is equal to or smaller than the predetermined threshold. The controller 28 may determine whether the determination device 1 is in the predetermined section based on a difference value between a maximum value and a minimum value, frequency analysis, etc. instead of the variance of acceleration. When acceleration measurement in a plurality of axis directions is possible, the controller 28 may determine whether the determination device 1 is in the predetermined section based on each of the acceleration or variance of a value obtained by adding an absolute value thereof.

When determining that the determination device 1 is not in the predetermined section in which the acceleration value needs to be recorded (S104: No), the controller 28 returns to processing of S100. When determining that the determination device 1 is in the predetermined section in which the acceleration value needs to be recorded (S104: Yes), the controller 28 goes to processing of S106.

The first calculator 40 may perform processing of S102 and S104. The controller 28 or the first calculator 40 may define, as the predetermined section, time in a predefined length from time to receive an operational input into the determination device 1 by the subject or a command input from outside, the operational input being made when the subject adopts a predetermined posture or when measurement starts. That is, the controller 28 or the first calculator 40 may perform control so that the first period starts when a predetermined signal is acquired from outside. Alternatively, the first period may be defined as time in a predefined length from time to acquire a predetermined signal from outside, the time being defined as predetermined time in which the determination device 1 is estimated to be in a stationary state based on the variance of acceleration, etc.

In step 106 (S106), the first calculator 40 causes, for example, the storage 24 to store (record) the acceleration measured by the first measurement unit 30.

In step 108 (S108), the first calculator 40 applies, to the acceleration recorded in the processing of S106, the above-described filtering process in order to eliminate variation in the acceleration caused by a respiration or temporary body movement of the subject. The first calculator 40 then calculates the body surface vector.

In step 110 (S110), the vector retention unit 42 retains the body surface vector calculated by the first calculator 40. A numerical value retained in the vector retention unit 42 is a representative value obtained by calculating an average or a median for every axis, for example, in time series information on the body surface vector calculated within the first period. A predefined value is set in the vector retention unit 42 as an initial value of the body surface vector.

In step 112 (S112), the second calculator 44 calculates, for example, difference information that indicates a difference between a body surface vector retained in the vector retention unit 42 and a body surface vector newly calculated by the first calculator 40. Specifically, the second calculator 44 calculates, for example, a cosine of an angle θ formed by a body surface vector V1 retained in the vector retention unit 42 and a body surface vector V2 newly calculated by the first calculator 40 according to the following equation 1.

cos(θ)=V1·V2/(|V1∥V2|) (1)

In step 114 (S114), the estimation unit 46 determines whether the difference information continuously indicates a difference equal to or greater than the predetermined value for time in the predefined length (second period). For example, the estimation unit 46 determines whether a state in which the cosine value calculated according to the above equation 1 is equal to or smaller than a predetermined threshold (the difference is equal to or greater than a predetermined value) continues for the second period. When determining that the state in which the cosine value is equal to or smaller than the predetermined threshold does not continue for the second period (S114: No), the estimation unit 46 returns to processing of S100. When determining that the state in which the cosine value is equal to or smaller than the predetermined threshold continues for the second period (S114: Yes), the estimation unit 46 goes to processing of S116.

In step 116 (S116), the estimation unit 46 estimates that an attached state of the determination device 1 changes. That is, when the difference information continuously shows a difference equal to or greater than the predetermined value for the second period, the estimation unit 46 determines that the attached state of the body unit with respect to the attachment surface changes.

The notification unit 48 then outputs information based on a determination result obtained by the estimation unit 46. For example, the determination device 1 continuously performs processing illustrated in FIG. 4. When determining that the attached state of the body unit 2 changes, the notification unit 48 outputs information indicating that the attached state of the body unit 2 changes for the purpose of instructing the subject, etc. to adjust an attachment position of the body unit 2. Accordingly, the determination device 1 measures and estimates a living body signal and body movement, etc. of the subject. When the attachment position of the body unit 2 is mispositioned, the determination device 1 outputs information showing the misposition.

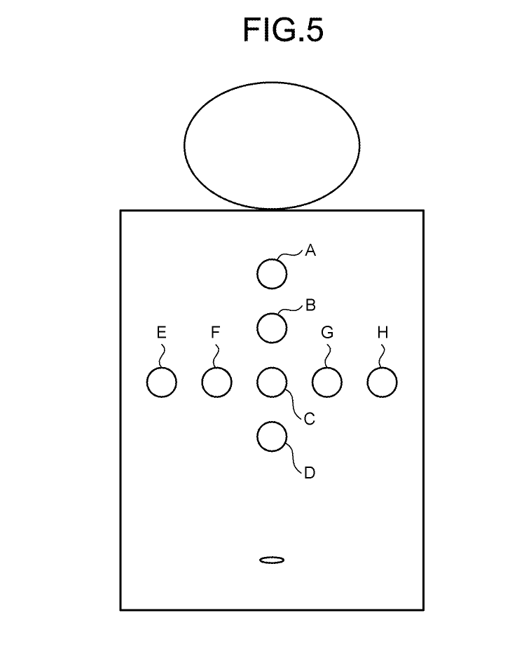

Next, the following describes a specific example of the difference information calculated by the second calculator 44 when the attached state (attachment position) of the determination device 1 changes. FIG. 5 is a schematic diagram schematically illustrating the attachment position (misposition) of the determination device 1 attached on a living body (human body). FIG. 6 is a graph illustrating a specific example of the difference information caused by the misposition of the attachment position illustrated in FIG. 5.

As illustrated in FIG. 5, for example, the body unit 2 is attached with the attachment part 200 made of an adhesive member at 2 cm intervals at eight points (point A to point H) on a trunk surface of the subject who is in a stationary supine-position state. The graph illustrating the difference information in FIG. 6 is a graph illustrating a cosine value of an angle formed by a body surface vector VA at chest point A and each of body surface vectors VB to VH at point B to point H respectively for every measurement count (0 to approximately 600 times).

In FIG. 6, the above-described cosine value decreases as the body unit 2 is distant from point A. The determination device 1 determines that the attached state changes when a state in which the cosine value is equal to or smaller than the predetermined threshold continues for the second period. For example, the determination device 1 determines that the attachment position of the body unit 2 is mispositioned with respect to the trunk surface of the subject when a state in which the cosine value is equal to or smaller than 0.989 (difference of the cosine value is equal to or greater than 0.011) continues for the second period (for example, period in which measurement is performed 500 times). That is, the determination device 1 has sufficient accuracy for determining that the attachment position of the body unit 2 is mispositioned even when, for example, the body unit 2 is mispositioned beyond 4 cm from point A (when distant beyond point C). The determination device 1 may be configured not to determine that the attached state changes even if the cosine value is equal to or smaller than the predetermined threshold when calculating difference information for the first time after mounted.

### Modification

Next, a modification of the determination device 1 will be described. In the modification of the determination device 1, after determination that the determination device 1 is in a predetermined section in which an acceleration value is recorded, the second calculator 44 calculates, for example, each of cosine values CX1, CY1, and CZ1 of angles formed by a vector retained in the vector retention unit 42 and axes of the first measurement unit 30. Furthermore, the second calculator 44 calculates each of cosine values CX2, CY2, and CZ2 of angles formed by a vector newly calculated by the first calculator 40 and each of axes of the first measurement unit 30.

The estimation unit 46 determines that the attached state of the body unit 2 changes when difference values between CX1 and CX2, between CY1 and CY2, between CZ1 and CZ2 are continuously equal to or greater than a predetermined threshold during the second period, the difference values being calculated by the second calculator 44.

The second calculator 44 may calculate at least one of an inner product, an angle, and a cosine value of the angle formed by a vector stored in the vector retention unit 42 and a measurement axis of each of acceleration in a plurality of directions as first information. The second calculator 44 may then calculate at least one of an inner product, an angle, and a cosine value of the angle formed by a vector newly calculated by the first calculator 40 and a measurement axis of each of acceleration in a plurality of directions as second information. The second calculator 44 may calculate a difference between the first information and the second information corresponding to the first information as difference information.

A determination program executed by the determination device 1 of the present embodiment, which may be provided as a computer program product, has a module configuration that includes the above-described each unit (first calculator 40, second calculator 44, and estimation unit 46). A function included in the determination device 1 may be configured with software and may be configured with hardware.

The above-described embodiment makes it possible to accurately determine that the attached state with respect to the attachment surface changes with a simple configuration because the determination device 1 determines that the attached state of the body unit with respect to the attachment surface changes when difference information showing a difference between inclination information stored in the storage and inclination information newly calculated by the first calculator continuously shows a difference equal to or greater than a predetermined value during the second period.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A determination device, comprising:
a first measurement unit fixed to a body unit to be attached to an attachment surface, and configured to measure acceleration in at least one direction;
a first calculator configured to calculate inclination information that indicates inclination of the attachment surface based on the acceleration measured by the first measurement unit during a first period;
a storage configured to store therein the inclination information calculated by the first calculator;
a second calculator configured to calculate difference information that indicates a difference between the inclination information stored in the storage and inclination information newly calculated by the first calculator;
a determination unit configured to determine that an attached state of the body unit with respect to the attachment surface changes when the difference information continuously indicates a difference equal to or greater than a predetermined value during a second period; and
an output unit configured to output information based on a determination result obtained by the determination unit.

2. The device according to claim 1, wherein
the first calculator is configured to calculate a vector that indicates the inclination of the attachment surface as the inclination information, and
the second calculator is configured to calculate at least one of an inner product, angle, and cosine value of the angle formed by the vector stored in the storage and a vector newly calculated by the first calculator as difference information.

3. The device according to claim 1, wherein
the first measurement unit is configured to continuously measure acceleration in a plurality of directions,
the first calculator is configured to calculate the vector that indicates the inclination of the attachment surface as the inclination information based on the acceleration in the plurality of directions, and
the second calculator is configured to
calculate at least one of an inner product, an angle, and a cosine value of the angle as first information, each of the inner product, the angle, and the cosine value being formed by the vector stored in the storage and a measurement axis of the acceleration in each of the plurality of directions,
calculate at least one of an inner product, an angle, and a cosine value of the angle as second information, each of the inner product, the angle, and the cosine value being formed by a vector newly calculated by the first calculator and a measurement axis of the acceleration in each of the plurality of directions, and
calculate a difference between the first information and the second information corresponding to the first information as difference information.

4. The device according to claim 1, wherein the output unit is configured to output information based on the determination result obtained by the determination unit through communication.

5. The device according to claim 1, wherein the first measurement unit is configured to continuously measure at least acceleration in a gravity direction.

6. The device according to claim 1, wherein
the attachment surface is on a surface of a living body, or in a position substantially identical to the surface of the living body, and
the device further comprises a controller configured to control so that the first period starts when the controller estimates that the living body is continuously in a stationary state based on the acceleration continuously measured by the first measurement unit or when the controller acquires a predetermined signal from outside.

7. The device according to claim 6, wherein the first calculator calculates the inclination information that indicates the inclination of the attachment surface, after performing a filtering process on the acceleration measured by the first measurement unit during the first period, the filtering process eliminating variation in the acceleration caused by a respiration or temporary movement of the living body.

8. The device according to claim 6, further comprising a second measurement unit configured to measure a living body signal, wherein the output unit is configured to further output information based on a measurement result obtained by the second measurement unit.

9. A determination method comprising:
measuring acceleration of a body unit to be attached to an attachment surface in at least one direction;
calculating inclination information that indicates inclination of the attachment surface based on the acceleration measured during a first period;
storing the calculated inclination information;
calculating difference information that indicates a difference between the stored inclination information and inclination information newly calculated;
determining that an attached state of the body unit with respect to the attachment surface changes when the difference information continuously indicates a difference equal to or greater than a predetermined value during a second period; and
outputting information based on a determination result obtained at the determining.

10. A computer program product comprising a computer-readable medium containing a program executed by a computer, the program causing the computer to execute:
calculating inclination information that indicates inclination of an attachment surface based on acceleration of a body unit to be attached to the attachment surface in at least one direction, the acceleration being measured during a first period;
storing the calculated inclination information;
calculating difference information that indicates a difference between the stored inclination information and inclination information newly calculated;
determining that an attached state of the body unit with respect to the attachment surface changes when the difference information continuously indicates a difference equal to or greater than a predetermined value during a second period; and
outputting information based on a determination result obtained at the determining.
